(19) United States
(12) Patent Application Publication
(10) Pub. No.: US 2022/0104545 A1
(43) Pub. Date: Apr. 7, 2022

(54) VAPORIZATION APPARATUS, SYSTEMS, AND METHODS

(71) Applicant: HEXO OPERATIONS INC., Ottawa (CA)

(72) Inventors: Max Alsayar, Ottawa (CA); Stephan Georglev, Salnt-Hubert (CA); Denis Keseris, Baie d'Urfe (CA)

(21) Appl. No.: 17/426,692

(22) PCT Filed: Feb. 1, 2020
(86) PCT No.: PCT/CA2020/050121

§ 371 Date: Jul. 29, 2021

Related U.S. Application Data
(60) Provisional application No. 62/800,164, filed on Feb. 1, 2019.

(51) Int. Cl. A24F 40/30; A24F 40/42; A24F 40/65; A24F 40/53; A24F 40/57; A24F 40/51; A24F 40/60

(52) U.S. Cl. CPC A24F 40/30; A24F 40/42; A24F 40/65; A24F 40/60; A24F 40/57; A24F 40/51; A24F 40/53

(57) ABSTRACT

The present disclosure generally relates to smart devices, apparatuses, and systems for vaporizing cannabinoid-containing substances, cannabis-terpene-containing substances, terpenes, and/or flavourings. These vaporization devices are configured to accept and make use of consumable cartridges containing cannabinoid-containing substances, cannabis-terpene-containing substances, flavourings, or any combinations thereof. According to an aspect, the disclosure relates to an apparatus for vaporizing cannabinoid-containing substances for inhalation. The apparatus may comprise a plurality of interfaces, each interface configured to communicate with cartridge containing a cannabinoid-containing substance suitable for vaporization; and a cartridge controller configured to control the operation of each cartridge individually.

### CROSS-REFERENCE TO RELATED APPLICATION

This application claims priority from U.S. Provisional Patent Application 62/800,164 filed on Feb. 1, 2019, which is incorporated herein by reference in its entirety.

### FIELD

This disclosure relates generally to cannabinoid-containing consumer products. In particular, the disclosure relates to systems, apparatus and methods for vaporizing cannabinoid-containing substances, cannabis-terpene-containing substances and flavourings.

### BACKGROUND

While the market for cannabinoid-containing consumer products is gaining momentum, historically, the clandestine nature of the cannabis industry has largely suppressed innovation and led to a market characterised by unsophisticated product development activity, as well underdeveloped consumer product safety and characterisation standards.

For example, traditional product descriptors, such as indica, hybrid and sativa, often have very little to do with a particular product's actual chemistry. As cannabinoid-containing products reach a growing number of mainstream consumers, these inadequacies will have significant adverse effects on consumers' and legislators' perceptions of the cannabis-related consumer product market.

For these and other reasons, there is a need for improvements in systems, apparatus and methods for vaporizing cannabinoid-containing substances, cannabis-terpene-containing substances and flavourings that will satisfy a growing demand and need for sophisticated consumption technologies that will afford consumers precise, consistent and tailored experiences and enable technology-driven and data-driven standardization of cannabis-based consumer products.

### SUMMARY

This Summary is provided to introduce a selection of concepts in a simplified form that are further described below in the Detailed Description. This Summary is not intended to identify key aspects or essential aspects of the claimed subject matter.

The present disclosure generally relates to smart devices, apparatuses, and systems for vaporizing cannabinoid-containing substances, cannabis-terpene-containing substances, terpenes, and/or flavourings. These vaporization devices are configured to accept and make use of consumable cartridges containing cannabinoid-containing substances, cannabis-terpene-containing substances, flavourings, or any combinations thereof.

According to an aspect, the disclosure relates to an apparatus for vaporizing cannabinoid-containing substances for inhalation. The apparatus may comprise a plurality of interfaces, each interface configured to communicate with a cartridge containing a cannabinoid-containing substance suitable for vaporization; and a cartridge controller configured to control the operation of each cartridge individually.

According to another aspect, the disclosure relates to a system comprising a vaporization device for producing vapor inhalable by a user. The vaporization device being configured to contain a plurality of vaporization substances that are stored separately from one another and vaporizable to produce the vapor. The system comprising: an input device configured to obtain information about the selected vaporization substances; and a processor configured to cause vaporization of the combination of the selected vaporization substances by the vaporization device such that the vapor includes the combination of the selected vaporization substances.

According to yet another aspect, the disclosure relates to an apparatus for producing vapor inhalable by a user, comprising: at least one interface, configured to communicate with a cartridge containing a vaporization substance containing a cannabis-derived compound and to receive cartridge information; a cartridge controller configured to control the operation of the cartridge; a communication interface configured to receive instructions from an input device; a processor configured to receive the instructions from the communication interface and to operate the cartridge controller for vaporization of the vaporization substance such that vapor is produced in accordance with the instructions, wherein the apparatus is configured to remain disabled until an activation signal is received from the input device, wherein the activation signal is issued by a mobile application accessing an authentication mechanism of the input device.

According to yet another aspect, the disclosure relates to an apparatus for producing a vapor inhalable by a user. The apparatus may comprise a plurality of interfaces, each interface configured to communicate with a cartridge containing a vaporization substance and to receive cartridge information. The apparatus may also comprise a cartridge controller configured to control the operation of each cartridge individually. The apparatus may further comprise a communication interface that may be configured to receive instructions from an input device. A processor may also form part of the apparatus, and be configured to receive instructions from the communication interface, receive the cartridge information from the interfaces, and operate the cartridge controller for vaporization of the vaporization substance such that vapor is produced in accordance with the instructions.

In an embodiment, the input device may be a mobile communication device.

In an embodiment, the instructions are generated on the mobile device using a mobile application relating to the apparatus.

In an embodiment, the processor may be configured to control the operation of the cartridge controller. The cartridge controller may be configured to control any one of:
a. the flow of vaporization substance through the one or more atomizers in the cartridge;
b. the power provided to the one or more atomizers in the cartridge;
c. the duty cycle of the one or more atomizers in the cartridge;
d. the vaporization temperature of the one or more atomizers in the cartridge;
e. the pre-heating temperature of the cartridge; or
f. a combination thereof.

In an embodiment, the apparatus may further comprise at least one volume sensor configured to sense the quantity of vaporization substance remaining in the cartridge. The volume sensor may generate content information conveying information relating to the quantity of vaporization substance remaining in the cartridge.

In an embodiment, the input device may be configured to obtain information about remaining amounts of vaporization substances, and the processor may be configured to communicate with a remote server to automatically order a cartridge containing a to vaporization substance determined to be in low quantity.

In another embodiment, the apparatus comprises a main body that may be configured to receive a plurality of cartridges containing vaporization substances. The cartridges may be disposed in a single insert, such that all cartridges are inserted into the apparatus simultaneously, also referred to as a multi-chamber cartridge.

In an embodiment, the processor may be configured to process the instructions for a vaporization formulation to determine whether the vaporization formulation can be achieved, based on the nature and the amount of vaporization substances remaining in the cartridges, or should be achieved, based on whether the formulation is allowable by law.

In an embodiment, the processor may be configured to disable the vaporization according to the instructions if the vaporization formulation cannot or should not be achieved by the vaporization device.

In an embodiment, the cartridge information received by the data interface may comprise information identifying the vaporization substance contained in the respective cartridge, information identifying the manufacturer of the respective cartridge, information identifying the distributor of the respective cartridge, information identifying the vendor of the respective cartridge.

In an embodiment, the apparatus may be further configured to prevent vaporization of the contents of a cartridge based on the cartridge information.

In an embodiment, the apparatus may be configured to prevent vaporization of the contents of a cartridge if the information identifying the manufacturer of the cartridge comprises information indicating that the cartridge was manufactured by a pre-determined manufacturer.

In an embodiment, the vaporization substances may be independently selected from a cannabinoid-containing substance, a terpene, a flavouring substance, or a combination thereof.

In an embodiment, one or more of the vaporization substances may comprise a THC-containing substance; the information comprises information about a level of THC in the one or more THC-containing substance; and the processor is configured to process the information about the level of THC in the THC-containing substance to determine whether the one or more THC-containing substance, alone or in combination with another vaporization substance, can or should be achieved by the vaporization device.

In an embodiment, the processor may be configured to disable the vaporization of the one or more THC-containing substance if the vaporization formulation cannot or should not be achieved by the vaporization device.

In an embodiment, the apparatus may be configured to provide cartridge content information to the input device.

In an embodiment, the apparatus may further comprise a Graphical User Interface (GUI) configured to implement a plurality of graphical control elements, the graphical control elements being configured to allow a user of the apparatus to control the cartridge controller to select the vaporization substance and/or the amounts of the vaporization substance to be vaporized by each cartridge.

In an embodiment, the graphical control elements may be further configured to allow a user to download, over a data-communication network, instructions for controlling the operation of the cartridge controller to set given amounts of the vaporization substance to be vaporized by each vaporization cartridge.

In an embodiment, the apparatus must be enabled by an activation signal received from the input device. The apparatus may remain disabled until such time as the mobile application running on the input device issues an activation signal to the apparatus; the activation signal in turn may be issued by the mobile application accessing the authentication mechanism of the input device, thus providing child-resistance features to the apparatus.

In use, a user selects a vaporization formulation on the input device. Without wishing to be limiting in any manner, the user may have been presented the formulation by the mobile application, or the user may have set or selected a vaporization formulation that has been at least partially set by the user interacting with the graphical control elements. The processor determines the required amounts of vaporization substance from each cartridge that is required to be vaporized in order to achieve the particular vaporization formulation selected by the user. The processor sends instructions to the cartridge controller, which controls the variables as described elsewhere herein, to produce the required amounts of vapor from each cartridge.

All features of exemplary embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

A detailed description of examples is provided below, with reference to the following drawings, in which:

FIG. 1 shows an isometric view of an example multi-chamber vaporization device;

FIG. 2 shows an isometric and partially exploded view of an example multi-chamber vaporization device;

FIG. 3 shows a cross-sectional view of the example multi-chamber vaporization device of FIGS. 1 and 2, along line A-A in FIGS. 1 and 2;

FIG. 4 shows an example cartridge for use with the example device shown in FIGS. 1 to 3;

FIG. 5 shows a functional block diagram of some elements of the circuitry comprised in the example cartridge of FIG. 4;

FIG. 6 shows a functional block diagram of some elements of the circuitry comprised in the example device shown in FIGS. 1 to 3;

FIG. 7 shows an example Graphical User Interface (GUI) of a communication device configured to control the example device shown in FIGS. 1 to 3;

FIG. 8 shows a high-level network diagram of an example data-communication system configured to be used with example devices for vaporizing cannabinoid-containing substances;

FIG. 9 shows a flowchart representing an example method of validating a cartridge for use with the example vaporization device of FIG. 1;

FIG. 10 shows a flowchart representing an example method of automatically reordering a cartridge for use with the example vaporization device of FIG. 1;

FIG. 11 shows a flowchart representing an example method of downloading a formulation recipe for use with the example vaporization device of FIG. 1; and

FIG. 12 shows a flowchart representing an example method of uploading a formulation recipe for use with the example vaporization device of FIG. 1.

In the drawings, exemplary embodiments are illustrated by way of example. It is to be expressly understood that the description and drawings are only for the purpose of illustrating certain embodiments and are an aid for understanding. They are not intended to be a definition of the limits of the invention.

### DETAILED DESCRIPTION

For illustrative purposes, specific example embodiments will be explained in greater detail below in conjunction with the figures. It should be appreciated, however, that the present disclosure provides many applicable concepts that can be embodied in any of a wide variety of specific contexts. The specific embodiments discussed are merely illustrative and do not limit the scope of the present disclosure. For example, embodiments could include additional, different, or fewer features than shown in the drawings. The figures are also not necessarily drawn to scale.

Unless otherwise defined, all technical and scientific terms used herein have the same meaning as commonly understood by a person of ordinary skill in the art to which the present invention pertains. As used herein, and unless stated otherwise or required otherwise by context, each of the following terms shall have the definition set forth below.

The present disclosure relates, in part, to vaporization devices for vaporization substances that include active substances ,such as one or more cannabinoids or nicotine. However, the vaporization devices described herein could also or instead be used for vaporization substances without an active substance.

The vaporization substance contained in a cartridge may be any vaporization substance, including those known in the art and suitable for vaporization. The particulars of cannabinoids and vaporization substances described herein are intended solely for illustrative purposes. Other embodiments are also contemplated. In one example, the vaporization substance may be a cannabinoid-containing substance, a terpene, a flavouring substance, or a combination thereof. The vaporization substances may be independently selected for each cartridge. The vaporization substance may be in the form of a solid, gel, wax, liquid, or liquid concentrate. Preferably, the vaporization substance comprises a liquid concentrate, such as an oil.

In some examples, the oil is a cannabis oil which contains one or more cannabinoids and is in liquid form at a temperature of 22±2° C.

As used herein, the term “cannabinoid” is generally understood to include any chemical compound that acts upon a cannabinoid receptor. Cannabinoids could include endocannabinoids (produced naturally by humans and animals), phytocannabinoids (found in cannabis and some other plants), and synthetic cannabinoids (manufactured artificially).

Examples of phytocannabinoids include, but are not limited to, cannabigerolic acid (CBGA), cannabigerol (CBG), cannabigerol monomethylether (CBGM), cannabigerovarin (CBGV), cannabichromene (CBC), cannabichromevarin (CBCV), cannabidiol (CBD), cannabidiol monomethylether (CBDM), cannabidiol-C4 (CBD-C4), cannabidivarin (CBDV), cannabidiorcol (CBD-C1), delta-9-tetrahydrocannabinol (Δ9-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-cis-iso tetrahydrocannabivarin, delta-8-tetrahydrocannabinol (Δ8-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2, 6-methano-2H-1-benzoxocin-5-methanol (OH-iso-HHCV), cannabiripsol (CBR), trihydroxy-delta-9-tetrahydrocannabinol (triOH-THC), cannabinol propyl variant (CBNV), and derivatives thereof.

Examples of synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, and quinolinyl esters.

A cannabinoid may be in an acid form or a non-acid form, the latter also being referred to as the decarboxylated form since the non-acid form can be generated by decarboxylating the acid form.

A vaporization substance may comprise a cannabinoid in its pure or isolated form or a source material comprising the cannabinoid. Examples of source materials comprising cannabinoids include, but are not limited to, cannabis or hemp plant material (e.g, flowers, seeds, trichomes, and kief), milled cannabis or hemp plant material, extracts obtained from cannabis or hemp plant material (e.g., resins, keif, waxes and concentrates), and distilled extracts or isolates. In some embodiments, pure or isolated cannabinoids and/or source materials comprising cannabinoids may be combined with water, lipids, hydrocarbons (e.g., butane), ethanol, acetone, isopropanol, or mixtures thereof.

In some examples, the cannabinoid is tetrahydrocannabinol (THC). THC is only psychoactive in its decarboxylated state. The carboxylic acid form (THCA) is non-psychoactive. Delta-9-tetrahydrocannabinol (Δ9-THC) and delta-8-tetrahydrocannabinol (Δ8-THC) produce the effects associated with cannabis by binding to the CB1 cannabinoid receptors in the brain.

In some examples, the cannabinoid is cannabidiol (CBD). The terms “cannabidiol” or “CBD” are generally understood to refer to one or more of the following compounds, and, unless a particular other stereoisomer or stereoisomers are specified, includes the compound “Δ2-cannabidiol.” These compounds are: (1) Δ5-cannabidiol (2-(6-isopropenyl-3-methyl-5-cyclohexen-1-yl)-5-pentyl-1,3-benzenediol); (2) Δ4-cannabidiol (2-(6-isopropenyl-3-methyl-4-cyclohexen-1-yl)-5-pentyl-1,3-benzenediol); (3) Δ3-cannabidiol (2-(6-isopropenyl-3-methyl-3-cyclohexen-1-yl)-5-pentyl-1,3-benzenediol); (4) Δ3,7-cannabidiol (2-(6-isopropenyl-3-methylenecyclohex-1-yl)-5-pentyl-1,3-benzenediol); (5) Δ2-cannabidiol (2-(6-isopropenyl-3-methyl-2-cyclohexen-1-yl)-5-pentyl-1,3-benzenediol); (6) Δ1-cannabidiol (2-(6-isopropenyl-3-methyl-1-cyclohexen-1-yl)-5-pentyl-1,3-benzenediol); and (7) Δ6-cannabidiol (2-(6-isopropenyl-3-methyl-6-cyclohexen-1-yl)-5-pentyl-1,3-benzenediol).

In some examples, the cannabinoid is a mixture of tetrahydrocannabinol (THC) and cannabidiol (CBD). The w/w ratio of THC to CBD a the vaporization substance may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1, or about 1000:1.

In some examples, a vaporization substance may include products of cannabinoid metabolism, including 11-hydroxy-Δ9-tetrahydrocannabinol (11-OH-THC).

These particulars of cannabinoids are intended solely for illustrative purposes. Other embodiments are also contemplated.

The vaporization substance may also be or include one or more terpene, or a mixture of terpenes. As used herein, the term “terpene” refers to both terpenes and terpenoids, which are a large and diverse class of organic compounds produced by a variety of cannabis plants. They include any organic compound derived biosynthetically from units of isoprene or derived through chemical synthesis. Typically known for their organoleptic effects, they are commonly used in the manufacture of edible products (e.g., gum, candies, mints) and fragrances. They are also recognized as entourage compounds meaning they modulate (e.g., enhance) the effects of the cannabinoids. At least some terpenes are expected to interact with, and potentiate the activity of, cannabinoids. Terpenes may be classified in many ways, such as by their sizes. For example, suitable terpenes may be selected from the group consisting of monoterpenes, diterpenes, triterpenes, hemiterpenes, sesquiterpenes, sesterterpenes, sesquarterpenes, and notisoprenoids.

Examples of suitable terpenes to formulate into the aroma composition may include aromadendrene, bergamottin, bergamotol, bisabolene, borneol, 4-3-carene, caryophyllene, cineole/eucalyptol, p-cymene, dihydroj asmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof. Additional examples of terpenes may include nerolidol, phytol, geraniol, alpha-bisabolol, thymol, genipin, astragaloside, asiaticoside, camphene, beta-amyrin, thujone, citronellol, 1,8-cineole, cycloartenol, and derivatives thereof. Further examples of terpenes are discussed in US Patent Application Pub. No. US2016/0250270.

In some examples, a vaporization substance may include a flavouring substance intended to add a flavour or a combination of flavours to the vapor produced by a vaporization device.

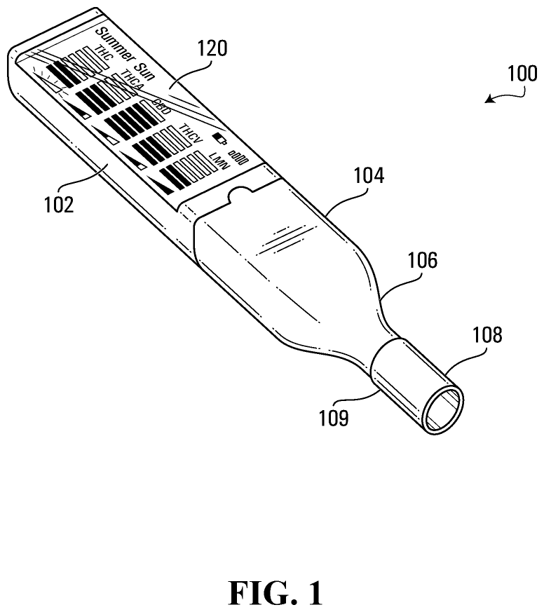

FIG. 1 is an isometric view of an example multi-chamber vaporization device 100. The vaporization device 100 has a multi-part body, with a main body 102 and a removable cover 104. The main body 102 and the cover 104 can be made from the same material(s) or different materials, including one or more of metals, plastics, elastomers and ceramics, for example. However, other materials can also or instead be used. The cover 104 tapers at 106 to a mouth-piece 108 in the example shown. The mouth-piece 108 can be made from the same material(s) as the remainder of the cover 104, and can even be integrated with the cover 104.

The mouth-piece 108 enables a user to inhale vapor produced by one or more of the atomizers 200ₓ described below. The mouth-piece 108 can include a mixing channel that is in fluid communication with the stems 202ₓ of the cartridges 130ₓ, and one or more output holes. A mixing channel could improve mixing of vapor from any or all of cartridges 130ₓ for inhalation through the output hole. Active and/or passive mixing, using any of various types of mixers can be used to mix vapor that is produced by multiple atomizers 200ₓ. The vaporization device 100 comprises a Graphical User Interface (GUI) 120, which will be described in more detail below.

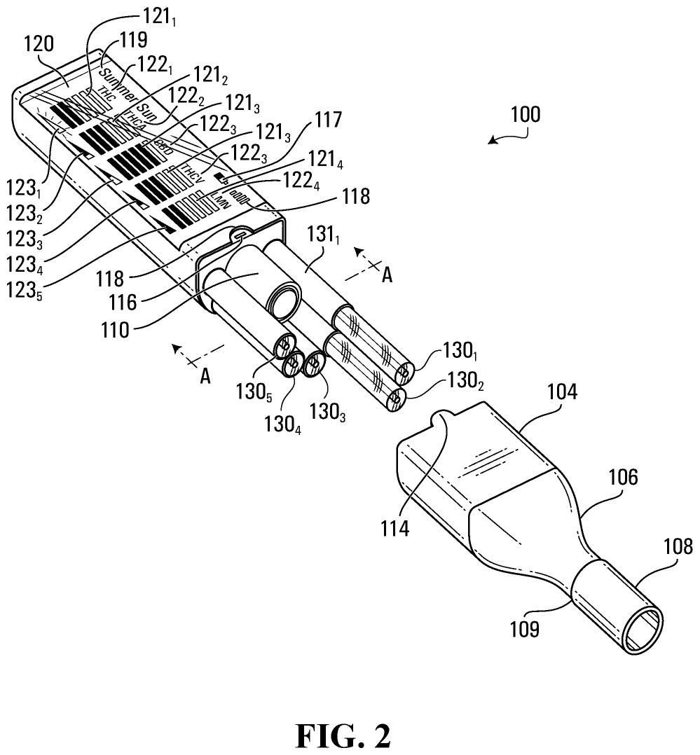

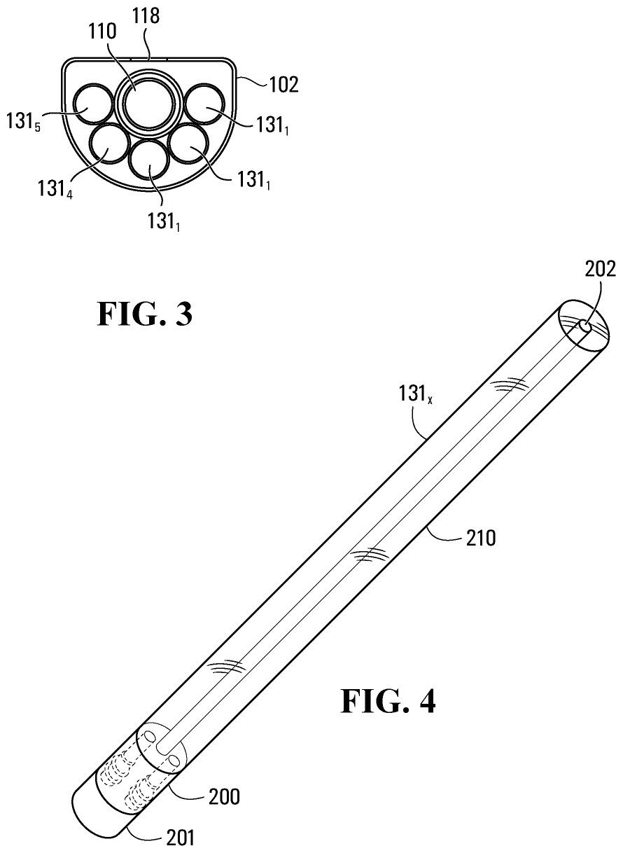

FIG. 2 is an isometric and partially exploded view of an example multi-chamber vaporization device, and FIG. 3 is a cross-sectional view of the example multi-chamber vaporization device along line A-A in FIG. 2. The main body 102 and the cover 104 include cartridge slots 131ₓ configured to receive cartridges 130ₓ containing substances to be vaporized, and battery compartment 110 configured to receive a battery to power the vaporization device 100. The battery compartment 110 can include single-use batteries or rechargeable batteries such as lithium-ion batteries. A releasable engagement enables replacement of single-use batteries and/or removal of rechargeable batteries for charging, for example. In some embodiments, rechargeable batteries could be recharged by an internal battery charger in the battery compartment 110 without removing them from the vaporization device 100. A charging port (not shown) could be provided in device 100.

The cartridge slots 131ₓ in the main body 102 are shown in FIG. 4, and the cover 104 may also include such compartments (not shown) in order to secure cartridges 130ₓ in place when the cover 104 is attached to the main body 102. Cartridges 130ₓ can also be secured to cartridge slots 131ₓ by other means, non-limiting examples of which include gendered connectors, threaded connectors and/or magnetic connectors.

In an embodiment, the cartridge for use in the apparatus may be refillable or replaceable. For example, in a refillable cartridge, the vaporization substance may be replaced or added to the one or more cartridge. This may be provided by a releasable engagement, such as a threaded engagement or other type of connection, or an abutment.

In the embodiment shown, the mouth-piece 108 engages with the remainder of the cover 104 at an engagement or connection 109. This engagement or connection 109 could be fixed, which might be preferable in embodiments in which the mouth-piece 108 is cylindrical as shown. In other embodiments, a rotatable or otherwise movable engagement or connection 109 may be preferred, so that a user can position the mouth-piece 108 in any preferred orientation relative to the main body 102 and/or the remainder of the cover 104.

The main body 102 can at least partially carry or otherwise support components such as the battery compartment 110 the GUI 120 and the cartridge slots 131ₓ as shown, and other components such as electrical contacts and/or circuitry. Similarly, the cover 104 can at least partially carry or otherwise support components such as the battery compartment 110, the GUI 120 and the cartridge slot 131ₓ, as well as the mouth-piece 108. The cartridge slots 131ₓ can be made, at least in part, from one or more materials such as tempered glass, plastics, metal alloys, and/or ceramics.

The cover 104 is removable or releasable from the main body 102. In the example shown in FIG. 1, a tab 114 on the cover 104 can be provided with a protrusion on its inner surface, to engage with a groove or slot 116 in the main body 102 when the vaporization device 100 is assembled or closed. This is an example of a releasable engagement between the main body 102 and the cover 104. The cover 104 can be removed, to install or remove cartridges 130ₓ and/or for cleaning the device 100 for example, by pulling the cover 104 away from the main body 102 with sufficient force to release the protrusion on the tab 114 from the slot or groove 116. Removal of the cover 104 in the embodiment shown can also or instead involve prying the tab 114 away from the slot or groove 116 to release the tab protrusion and allow the cover to be removed. The main body 102 can include a structure 118 to accommodate the tab 114, so that the outer surface of the tab is flush with the outer surface of the main body when the device 100 is assembled.

In operation, one or more batteries inside the battery compartment 110 provide power to the electronics of the vaporization device 100, as well as one or more cartridges 130ₓ, which vaporize one or more vaporization substances individually and/or one or more mixtures of vaporization substances.

The battery compartment 110 and/or the cartridge slots 131ₓ can be accommodated entirely within the main body 102, in which case the cover 104 need not include compartments to receive part of the battery compartment 110 and/or each cartridge slot 131ₓ.

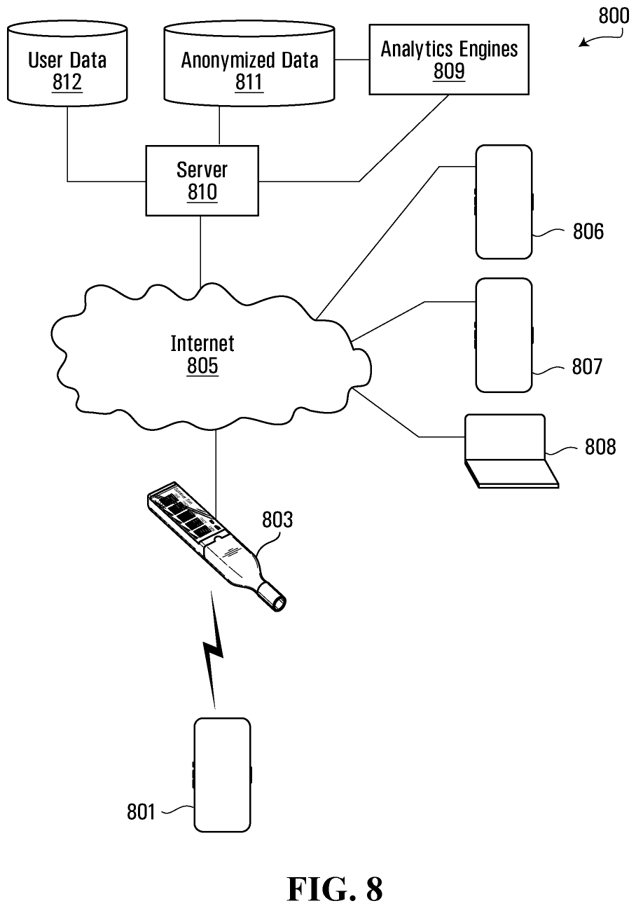

As mentioned above, in some embodiments, the device 100 further comprises a graphical user interface (GUI) 120, as shown in FIGS. 1 and 2. In some embodiments, the device 100 is battery operated, and the GUI 120 comprises a battery indicator 117 configured to indicate the relative charge left in the battery. The GUI 120 also comprises a signal indicator 118, conveying information to a user indicating the data-communication connection strength of the communication interface 604, with any device to which it is connected. The GUI 120 also comprises a title indicator portion 119 configured to show a title of a particular recipe (also referred to herein as “vaporization formulation”) that has been chosen by a user of the device, the recipe being indicative of a given vapor formulation comprising specific relative amounts of vapor to be produced by the cartridges 130ₓ. In some embodiments, the GUI 120 may also comprise a descriptor indicator portion (not shown) configured to show a description of a particular recipe or vaporization formulation that has been chosen by a user of the device. In some embodiments, the GUI 120 also comprises graphical control elements (not shown) allowing a user to control operation of the device. Non-limiting examples of graphical control elements include graphical control element configured to allow a user to cycle through recipes that are stored in memory 603, and graphical control elements configured to allow a user to request more recipes from server 810 or device 801, as described in more detail below with reference to FIG. 8.

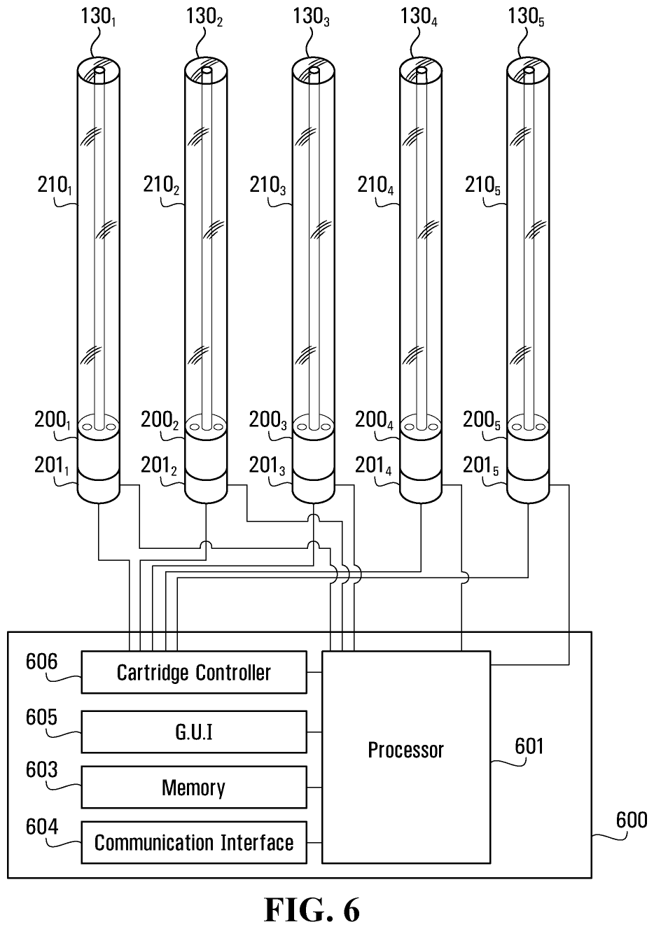

The GUI 120 also comprises cartridge information indicators 121ₓ, showing the contents of the cartridges, e.g. tetrahydrocannabinol (THC), tetrahydrocannabinolic acid (THCA), cannabidiol (CBD), delta-9-tetrahydrocannabivarin (THCV) and limonene (LMN), as well as the relative composition indicators 122ₓ showing the relative amounts of vapor that the cartridges are set to dispense through mouthpiece 108 by the device using the system shown in FIG. 6 and described below.

In some embodiments, the GUI 120 is a touch screen, and the relative amounts can be set by a user by setting the relative composition indicators 122ₓ manually. In some embodiments, the GUI 120 comprises cartridge volume indicators 123ₓ configured to convey information relating to the amount of vaporization substance remaining in each cartridge. In some examples, the cartridge volume indicators 123ₓ are configured to alert a user when the amount of vaporization substance in a given cartridge falls below a threshold. In some embodiments, the processor is configured to generate a low volume alert signal that can optionally be communicated to the user. As represented in FIG. 4, cartridge volume indicator 123₁ is blinking/flashing because its associated cartridge (e.g. THC-based vaporization oil) is almost empty.

It should be appreciated, however, that the example device 100 is solely for the purpose of illustration. Compartments could be implemented in any of various ways, and not only as the bores shown at 311, 313 in FIG. 4. Multiple engagement structures such as the tab 314 and the slot or groove 316 could be provided. Other types of connection or engagement between a main body and a cover, such as a magnetic connection, are also possible. Different shapes or layouts could also be implemented, to have a central air channel with compartments or structures to accommodate chambers around the central air channel, for example. A multi-chamber vaporization device with a hexagonal cross-sectional shape, for example, could accommodate six cartridges or chambers around a central air channel or mixing channel. At least certain shapes can be suitable for other types of releasable engagement between a main body and a cover, such as a threaded engagement for a cylindrical vaporization device.

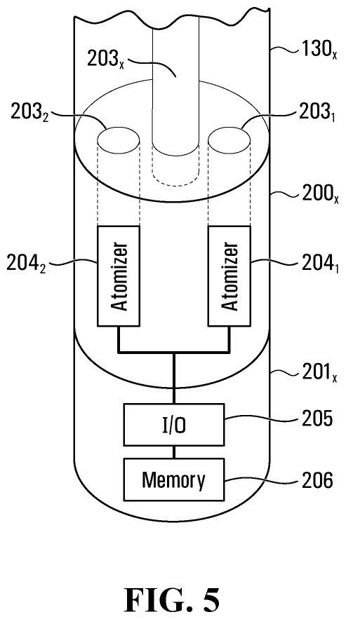

FIGS. 4 and 5 show an example cartridge 130ₓ for use with an example vaporization device 100. In some examples, the cartridge 130ₓ comprises a vaporization chamber 200, a connector 201 and a vaporization reservoir 210. The vaporization reservoir 210 can be made of any suitable material, including, but not limited to, glass, ceramic, and plastic. In some examples, the connector 201 is configured to mechanically and/or magnetically cooperate with a corresponding connector forming part of device 100. In some examples, the connector 201 and the device interface are configured to interlock mechanically.

In some examples, the connector 201 is configured to electronically cooperate with a corresponding device interface forming part of the device 100. In particular, the connector 201 may comprise circuitry including an Input/Output (I/O) port 205 configured to send and receive power and signals from the device 100, and a memory 206 for storing information relating to the cartridge 130ₓ. In some examples, the cartridge 130ₓ comprises a Radio Frequency Identification (RFID) tag configured to convey information related to the cartridge 130ₓ. In other examples, the cartridge 130ₓ comprises other mechanical or electronic means for conveying information related to the cartridge.

Examples of information relating to the cartridge include, but are not limited to, the contents of the cartridge 130ₓ (e.g. the identity of any cannabinoids present in the contents of the cartridge, as well as the amount by volume of the same), the identity of the manufacturer, the identity of the distributor and/or the identity of the vendor of the cartridge 130ₓ, or information relating to the shelf life of the contents of the cartridge 130ₓ.

In some embodiments, the cartridge 130ₓ comprises a cartridge memory 206 for conveying information relating to the cartridge. In the non-limiting example of FIG. 5, the cartridge memory 206 is a passive flash memory. Advantageously, this allows for the cartridge 130ₓ to be powered by the device 100, once connected, instead of comprising its own power source.

In some embodiments, cartridge 130ₓ comprises a volume sensor configured to measure the remaining quantity of vaporization substance present in the reservoir 210. Examples of volume sensors include but are not limited to load cells, strain gauges, capacitance level sensors, ultrasonic level sensors, optical level switches or conductive sensors. The volume sensors are further configured to emit a signal indicative of the remaining quantity of liquid or powder present in the reservoir, which signal can be communicated to the device 100 by way of the cartridge I/O 205, or can be stored in memory 206 for access by the device 100. Information relating to the remaining quantity of vaporization substance present in the reservoir 210 can therefore be saved to memory 206 each time the vaporization substance is vaporized. Alternatively, the information relating to the remaining quantity of vaporization substance present in the reservoir can be saved to memory 206 be retrieved by the device 100 as set intervals of time.

In other embodiments, a signal indicative of the remaining quantity of liquid or powder present in the reservoir can be generated by a volume sensor forming part of the device 100. In some examples, each cartridge slot 131ₓ comprises a volume sensor for determining the remaining quantity of vaporization substance present in the reservoir associated with the cartridge inserted in the respective cartridge slot 131ₓ. In some embodiments, the volume sensor associated with the each cartridge slot 131ₓ is configured to optically determine the level of substance remaining in the reservoir associated with the cartridge inserted in the respective cartridge slot 131ₓ.

An atomizer chamber 200 is provided at the base of each cartridge 130ₓ. The atomizer chamber 200 comprises one or more vaporization substance inlets 203ₓ, and one or more atomizers 204ₓ. The atomizers 204ₓ may also be referred to as a heating elements, cores, or ceramic cores. The atomizer chamber 200 engages, and can be couple with, and is in fluid communication with, the stem 202 of the cartridge 130ₓ via an engagement 132.

In general, the atomizers 204ₓ convert the vaporization substance in the chamber 204 into a vapor, which a user draws from the vaporization device 100 through the stem 202 and the cover 104. Vaporization liquid, for example, could be drawn into the atomizers 204ₓ through the vaporization substance inlets 203ₓ and a wick. In some embodiments, the atomizers 204ₓ include a heating element, such as a resistance coil around a ceramic wick, to perform the conversion of vaporization substance into vapor.

The connector 201 generally includes circuitry to supply power from the device 100 to the atomizers 204ₓ. For example, the connector 201 can include electrical contacts that connect to corresponding electrical contacts in the cartridge slot 131ₓ. The connector 201 can further include electrical contacts that connect to corresponding electrical contacts in each atomizer 204ₓ. In some embodiments, the connector 201 can reduce, regulate or otherwise control the power/voltage/current output from the device 100. However, this functionality could also or instead be provided by the device 100 itself.

In some embodiments, and with particular reference to FIG. 5, in order to ensure that the required amounts of vapor are produced by device 100, the cartridge 130ₓ is configured to allow several parameters of its operation to be controlled by the cartridge controller 606, as described in more detail below. In particular, the cartridge 130ₓ is configured to allow flow control, power control, duty cycle control and pre-heating.

In some embodiments, control can be achieved by adjusting the power delivered to the heating element. The power could be regulated in order to ensure optimal vaporization of the material to be vaporized, for example. In some embodiments, the cartridge is arranged to pre-heat its contents to control the flow rate by way of changing the viscosity the material to be vaporized. In some embodiments, the cartridge may have an electro-mechanical valve to control the flow of the material to be vaporized. In some embodiments, in addition to electrical control, a temperature sensor in or near the heating element/atomizer is provided to give active feedback to a temperature controller for more precise temperature control. In particular if the battery is operated by airflow control, this can be used to ensure that a strong inhalation does not overheat the material to be vaporized and lead to over saturation of the atomizer and/or leaking.

In some embodiments, different voltages and/or currents can be supplied to different atomizers 200ₓ to enable each atomizer to provide different vaporization temperatures. This type of power control can be considered a form of vaporization temperature control, and can be provided through one or more user input devices and/or GUI 605, and/or based on sensing the types of cartridges 130ₓ currently installed in the device 100. In general, the voltage, current, and/or power supplied to any one or more of the atomizers 200ₓ can be adjusted based on the vaporization substance to be vaporized in each atomizer. The voltage, current, and/or power supplied to any one or more of the atomizers 200ₓ can also or instead be adjusted based on a desired flow or quantity of vapor produced by that atomizer, which can be selected or otherwise controlled using one or more user input devices and/or GUI 605, for example.

The flow of vapor from any one or more of the atomizers 200ₓ can be controlled by controlling valves (not shown) which are in fluid communication with the atomizers through respective air channels (not shown).

In some embodiments, and with further reference to FIG. 6, when a user selects a particular recipe, or sets the relative compositions of a vaporization formulation, the processor 601 receives the formulation instructions form the GUI 120 or the memory 603 and/or any other device such as, but not limited to, devices 806, 807, 808 and 801 shown in FIG. 8, via the communication interface 604. Once received, the processor 601 determines the required amounts of cannabinoids and flavoring that each cartridge is required to vaporize in order to achieve the particular vapor formulation. The processor 601 and cartridge controller 606 could be implemented, for example, using hardware, firmware, one or more components that execute software stored in one or more non-transitory memory devices (not shown), such as a solid-data memory device or a memory device that uses movable and/or even removable storage media. Microprocessors, Application Specific Integrated Circuits (ASICs), Field Programmable Gate Arrays (FPGAs), and Programmable Logic Devices (PLDs) are examples of processing devices that could be used to execute software.

For example, if a particular composition requires 40 mg of THC, 30 mg of CBD, 20 mg of limonene, 40 mg of citrus flavour, the processor 601 determines the contents of the cartridges 130ₓ by, for example, querying memory 603 for relevant cartridge information. Upon receipt of the cartridge information, the processor can determine whether the requested vapor formulation can be or should be achieved, based on the nature and the amount of vaporization substances remaining in the cartridges 130ₓ, and/or based on whether the formulation is allowable by local law. For example, if cartridge 130₅ contains limonene, cartridge 130₁ contains THC, and cartridge 130₃ contains CBD, it will be possible to produce the required formulation, provided that each cartridge has sufficient amounts of vaporization substance, and that the concentrations of THC, CBD and limonene are sufficient to produce the desired vapor formulation, given the remaining amounts of vaporization substances.

Once a determination is made that the device 100 can produce the vapor, the processor 601 sends instructions to the cartridge controller 606 which controls the cartridge operation, as described above, to ensure that the required amounts of vapor are produced by each cartridge.

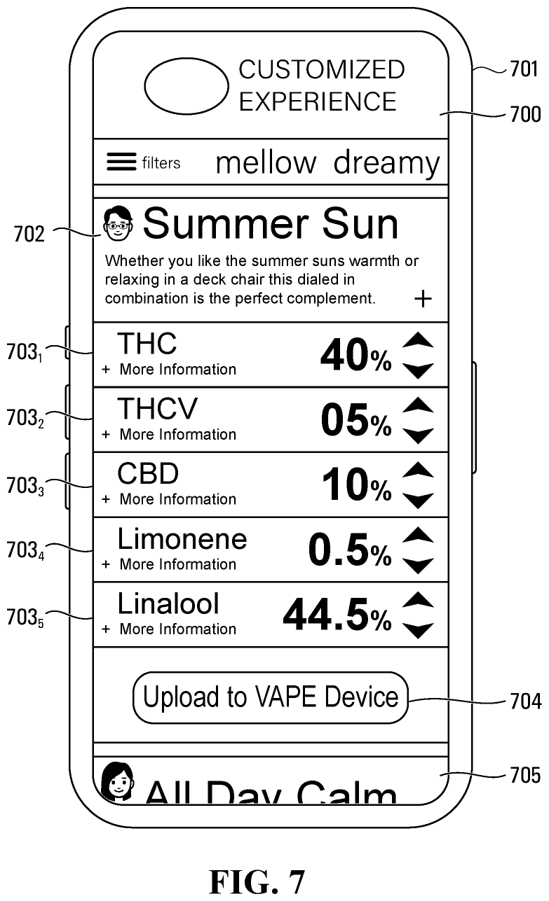

As shown in FIG. 7, in some embodiments, the mobile device 701, 801 is configured to implement a Graphical User Interface (GUI) 700 allowing a user to control device 100, 803. In some embodiments, the GUI is implemented by a mobile application (or “app”). In some embodiments, the GUI 700 comprises a title and descriptor area 702, in which the title of a vaporization recipe is shown, and a description of the recipe is shown. In some embodiments, the description of the recipe comprises information conveying a feeling or experience which may be achieved by consumption of the vaporization recipe. In some embodiments, the description of the recipe comprise information conveying an appropriate activity during which the vaporization recipe can be consumed. In some embodiments, the description of the recipe comprises information conveying an appropriate setting in which the vaporization recipe can be consumed.

The GUI 700 also comprises graphical control elements 703ₓ which serve to convey information about a vaporization formulation to a user, and to allow a user to change the compositions of a vaporization formulations, by interacting with the graphical control elements to dial the amount of any cannabinoid-containing vapor (or other additive or flavoring) either up or down, by pressing the appropriate graphical control element arrows. In some embodiments, the graphical control elements 703ₓ also allow a user to seek more information (e.g. be pressing the “+More Information” graphical control element) about a particular cannabinoid-containing vaporization substance (or other additive or flavoring).

In some embodiments, the GUI 700 comprises a graphical control element 704 allowing a user to instruct the mobile device 701, 801 to upload a set formulation to device 100, 803. In some examples, the set formulation is a vaporization formulation or recipe that has been presented to the user by the mobile application. In other examples, the set formulation is a vaporization formulation or recipe that has been at least partially set by the user interacting with the graphical control elements.

In some embodiments, the device 100, 803 can be enabled by using the authentication mechanism of the mobile device 701, 801. For example, the device 100, 803 can be disabled until such time as the mobile application running on the mobile device 701, 801 issues an activation signal to the device 100, 803. The activation signal in turn can be issued by the mobile application accessing the authentication mechanism of the mobile device (e.g. fingerprint recognition mechanism, PIN code entry and/or facial recognition mechanism) in order to authenticate the user. This method can also be used to ensure child-resistance.

In some embodiments, the title and descriptor area 705 of the next vaporization formulation accessible by the mobile application can be seen at the bottom of the GUI 700, and the GUI is configured to allow the user to scroll up and/or down to browse through various vaporization formulations. In some embodiments, the mobile application is configured to collect formulation browsing information indicative of the formulations browsed by the user.

FIG. 8 shows a high-level network diagram of an example data-communication system configured to be used with example device 100, 803. In some embodiments, device 100, 803 is configured to be controlled by a local communication device 701,801. In some embodiments, the local communication device is a mobile communication device 701,801, such as a smartphone or tablet.

In some embodiments, device 100, 803 and/or mobile device 701, 801 are configured to communication with other devices 806, 807, 808 over the internet 805 in order to share transmit and receive vaporization formulations, cartridge information and other information related to the use of device 100, 803. In some embodiments, device 100, 803 are also configured to communicate with server 810, which may be a server relating to the manufacturer, distributor and/or vendor of the cartridges containing the cannabinoid-containing vaporization substances or other additives and/or device 100, 803. In some embodiments, use information generated by device 100, 803 and/or mobile device 701, 801 can be sent to server 810. Use information can include, but is not limited to, cartridge information, cartridge content information, formulation browsing information, the number of times a device 100, 803 has been used to dispense a formulation, the compositions of the vapor formulations dispensed by a device 100, 803, the geographic location of a device 100, 803, the registered owner/user of a device. In some embodiments, the server 810 can store user data in secured database 812, and anonymized use and/or user data, and other related data in database 811. Examples of anonymized user data are geographical location of devices, the time of day that certain formulations have been dispensed, etc. In some embodiments, the anonymized data can be run through various analytics engines 809 in order to determine user preference information for certain vaporization substances, cartridges, devices 100, 803 and/or compositions. In some embodiments, the user preference information can be used to provide a user with recommendations comprising recommended vaporization formulations, vaporization substances, flavouring additives and/or other additives. In some embodiments, the recommendations can be provided to the user by way of device 100, 803. In some embodiments, the recommendations can be provided to the user by way of the mobile application running on mobile device 701, 801.

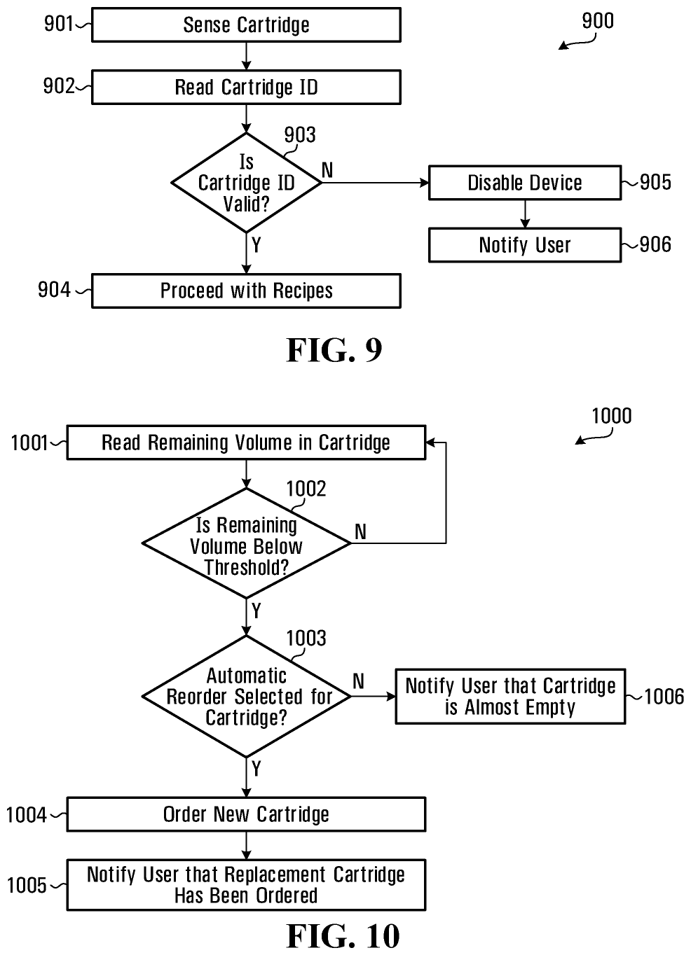

With particular reference to FIG. 9, a method of validating a cartridge for use in the device 100 is shown. At step 901, when a user inserts a cartridge 130ₓ in a slot 131ₓ of the device 100, the device senses the presence of the cartridge 130ₓ. Sensing (or detecting) the presence of the cartridge 130ₓ can be accomplished any number of ways including, but not limited to, detecting the presence of the connector 201ₓ by any suitable electromechanical, electronic or optoelectronic means. Once the device 100 detects the presence of a cartridge 130ₓ, the device reads the cartridge information stored in memory 206 in order to derive cartridge information therefrom. As will be appreciated, this can be done through I/O 205. In some examples, the device 100 comprises a barcode reader for reading a barcode (not shown) on each cartridge 130ₓ. In some examples, the device comprises an RFID reader for reading an RFID tag (not shown) on each cartridge 130ₓ.

Once the cartridge information is read by the device 100, a determination is made as to whether or not the cartridge information of the cartridge is valid at step 903. In some examples, the cartridge information includes information relating to the contents of the cartridge 130ₓ and/or the identity of the manufacturer, distributor and/or vendor of the cartridge 130ₓ. If a determination is made that the cartridge information is valid, then the device allows a user to use the cartridge 130ₓ with device 100, as described above. If, on the other hand, a determination is made that the cartridge information is invalid, then the device is configured to prevent a user from using the cartridge 130ₓ with device 100.

Examples of valid cartridge information include, but are not limited to, information indicating that a cartridge contains a particular product in a particular concentration (e.g. THC of a particular concentration), or that the cartridge 130ₓ was made by a given manufacturer, distributed by a given distributor, and/or sold by a given vendor. Examples of invalid cartridge information include, but are not limited to, information indicating that a cartridge contains a particular product in a particular concentration that is not allowed by one of the settings of the device 100 (e.g. THC of a particular concentration that is above a certain threshold), or if the cartridge was not made by a particular manufacturer, not distributed by a particular distributor, and/or not sold by a particular vendor.

At step 905, after a determination is made that the cartridge is not valid (for any one or more of the reasons set out above, or any other reason), the device disables the cartridge 130ₓ by way of the cartridge controller 606 so as to prevent the user from vaporizing the contents of the cartridge 130ₓ using device 100. Then at step, 906, the user can be notified of the fact that the device 100 has detected an invalid cartridge 130ₓ. Alternatively, at step 904, after a determination is made that the cartridge is valid (for any one or more of the reasons set out above, or any other reason), then the device does not disable the cartridge 130ₓ so as to allow the user to vaporize the contents of the cartridge using device 100.

FIG. 10 shows a flowchart representing an example method of automatically reordering a cartridge 130ₓ for use with an example vaporization device 100. At step 1001, the device 100 reads the remaining volume of vaporization substance in a cartridge 130ₓ, in accordance with the above. At step 1002, a determination is made as to whether the remaining volume of vaporization substance is below a certain threshold. If the remaining volume is below a certain threshold, a determination is made as to whether the device 100 is set to automatically reorder cartridges at step 1003. In some examples, the user will have enabled this feature in order to avoid running out of vaporization substances. If the automatic reorder feature is not enabled, the device can notify the user that the cartridge is almost empty at step 1006. If the automatic reorder feature is enabled, the device can use the communication interface 604 to contact a reorder server to order a new cartridge, at step 1004. In some embodiments, reorder server is server 810. Finally, at step 1005, the device 100 can notify the user that a replacement cartridge 130ₓ has been ordered at step 1005. In some embodiments, the device can notify the user by way of GUI 605 and/or GUI 700.

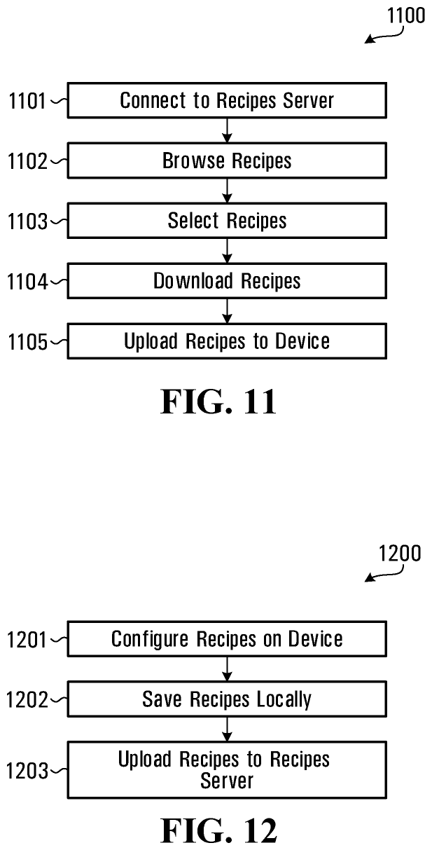

FIG. 11 shows a flowchart representing an example method of downloading a vaporization recipe for use with an example device 100. At step 1101, device 100 or mobile device 803 connects to a formulation server configured to provide data indicative of various vaporization formulations. In some embodiments, the formulation server is server 810. At step 1102, a user can browse formulations using the GUI of device 100 or the GUI 700 of the device 701. At step 1103, the user can select a formulation using one of the graphical control elements of the GUI of device 100 or the GUI of device 701, 801. After selecting the vaporization formulation, the formulation data can be downloaded to device 100 or device 701, 801 at step 1104. The formulation data comprises any data required for the device 100 to vaporize a given formulation from a plurality of cartridges. Optionally, if the formulation data is downloaded to device 701, 801, the formulation data can then be uploaded to device 100 at step 1105.

FIG. 12 shows a flowchart representing an example method of uploading a vaporization recipe for use with an example device 100 for vaporizing. At step 1201, a vaporization formulation can be configured by a user on device 100, or can be configured on the device 701, 801 for use on device 100. At step 1202, the formulation can be saved locally on device 100 or device 701, 801. At step 1203, the configured formulation can be uploaded from device 100 or device 701, 801 to a formulation server. In some embodiments, formulation server is server 810.

The skilled reader will readily recognize that steps of various above-described methods can be performed by programmed computers. Herein, some embodiments are also intended to cover program storage devices, e.g., digital data storage media, which are machine or computer readable and encode machine-executable or computer-executable programs of instructions, wherein said instructions perform some or all of the steps of said above-described methods. The embodiments are also intended to cover computers programmed to perform said steps of the above-described methods.

It should be appreciated by those skilled in the art that any block diagrams herein represent conceptual views of illustrative circuitry embodying the principles disclosed herein. Similarly, it will be appreciated that any flow charts and scripts and the like represent various processes which may be substantially represented in computer readable medium and so executed by a computer or processor, whether or not such computer or processor is explicitly shown.

It should be appreciated that the drawings and description herein are intended solely for illustrative purposes, and that the present invention is in no way limited to the particular example embodiments explicitly shown in the drawings and described herein.

What has been described is merely illustrative of the application of principles of embodiments of the present disclosure. Other arrangements and methods can be implemented by those skilled in the art.

While the present invention has been described with reference to specific features and embodiments thereof, various modifications and combinations can be made thereto without departing from the invention. The description and drawings are, accordingly, to be regarded simply as an illustration of some embodiments of the invention as defined by the appended claims, and are contemplated to cover any and all modifications, variations, combinations or equivalents that fall within the scope of the present invention. Therefore, although the present invention and potential advantages have been described in detail, various changes, substitutions and alterations can be made herein without departing from the invention as defined by the appended claims. Moreover, the scope of the present application is not intended to be limited to the particular embodiments of any process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1.-59. (canceled)

60. An apparatus for producing vapor inhalable by a user, comprising:
a. a plurality of interfaces, each interface configured to communicate with a cartridge containing a vaporization substance and to receive cartridge information;
b. a cartridge controller configured to control operation of each cartridge individually;
c. a communication interface configured to receive instructions for a vaporization formulation from an input device; and
d. a processor configured to perform the steps of:
i. receiving the instructions from the communication interface,
ii. receiving the cartridge information from the interfaces,
iii. determining from the instructions and from the cartridge information whether the vaporization formulation is permitted to be produced, and
iv. operating the cartridge controller for vaporization of the vaporization substance such that vapor is produced in accordance with the instructions if the determination is made that the vaporization formulation is permitted to be produced.

61. The apparatus of claim 60, wherein the input device is a mobile device and the instructions are generated on the mobile device using a mobile application relating to the apparatus.

62. The apparatus of claim 60, wherein the processor is configured to prevent the vaporization if the determination is made that the vaporization formulation is not permitted to be produced.

63. The apparatus of claim 60, wherein determining whether the vaporization formulation is permitted to be produced comprises detecting a presence of each cartridge at its interface

64. The apparatus of claim 60, wherein determining whether the vaporization formulation is permitted to be produced comprises receiving one or both of a shelf life and a number of times the apparatus has been used to dispense the vaporization formulation.

65. The apparatus of claim 60, wherein determining whether the vaporization formulation is permitted to be produced comprises determining whether the vaporization formulation is allowable by local law.

66. The apparatus of claim 60, wherein the cartridge information comprises at least one of information identifying the vaporization substance contained in the respective cartridge, information identifying a manufacturer of the respective cartridge, information identifying a distributor of the respective cartridge, and information identifying a vendor of the respective cartridge.

67. The apparatus of claim 66, wherein determining whether the vaporization formulation is permitted to be produced comprises receiving a number of times the identified vaporization substance contained in the respective cartridge has been used.

68. The apparatus of claim 66, wherein determining whether the vaporization formulation is permitted to be produced comprises comparing a concentration of the identified vaporization substance contained in the respective cartridge to a permitted concentration.

69. The apparatus of claim 60, wherein the vaporization substances are independently selected from a cannabinoid-containing substance, a terpene, a flavouring substance, or a combination thereof.

70. The apparatus of claim 60, wherein one or more of the vaporization substances comprises a CBD-containing substance or a THC-containing substance.

71. The apparatus of claim 60, further comprising a Graphical User Interface (GUI) configured to implement a plurality of graphical control elements, the graphical control elements being configured to allow a user of the apparatus to select the vaporization formulation for production of vapor by the apparatus.

72. The apparatus of claim 60, wherein the processor is further configured to receive the instructions from the input device that have been downloaded by a user over a data-communication network.

73. The apparatus of claim 72, wherein the vaporization formulation instructions are downloaded from cartridge's manufacturer, a distributor or a vendor's server or website.

74. A system comprising a vaporization device for producing vapor inhalable by a user, the vaporization device being configured to contain a plurality of vaporization substances that are stored separately from one another and vaporizable to produce the vapor, the system comprising:
a. an input device configured to obtain information about selected vaporization substances; and
b. a processor configured to process the information about the selected vaporization substances to determine whether producing a combination of the selected vaporization substances is permitted, and to cause vaporization of the combination of the selected vaporization substances by the vaporization device such that the vapor includes the combination of the selected vaporization substances in response to a determination that producing the combination of the selected vaporization substances is permitted.

75. The system of claim 74, wherein the processor is configured to disable the vaporization of the selected vaporization substances if the combination of the selected vaporization substances is determined to not be permitted to be vaporized by the vaporization device.

76. The system of claim 74, wherein the processor is configured to determine whether producing the combination of the selected vaporization substances is permitted comprises receiving one or both of a shelf life and a number of times the vaporization device has been used to dispense the combination.

77. The system of claim 74, wherein the processor is configured to determine whether the combination of the selected vaporization substances is permitted to be produced comprises determining whether the combination of the selected vaporization substances is allowable by local law.

78. The system of claim 74, wherein the information about the vaporization substances comprises at least one of information giving an identification, a volume remaining, or concentration of the vaporization substance contained in the respective cartridge.

79. The system of claim 74, wherein the vaporization substances are independently selected from a cannabinoid-containing substance, a terpene, a flavouring substance, or a combination thereof.

80. The system of claim 74, wherein: the information comprises information about at least one manufacturer of the selected vaporization substances; and the processor is configured to process the information about the at least one manufacturer of the selected ones of the vaporization substances to determine whether the combination of the selected ones of the vaporization substances can or should be vaporized by the vaporization device.

81. The system of claim 74, wherein the vaporization device comprises a body configured to receive a plurality of cartridges containing independently selected vaporization substances.

82. The system of claim 74, wherein the vaporization device comprises the input device and the processor.

83. An apparatus for producing vapor inhalable by a user, comprising:
a. at least one interface, configured to communicate with a cartridge containing a vaporization substance containing a cannabis-derived compound and to receive cartridge information;
b. a cartridge controller configured to control operation of the cartridge;
c. a communication interface configured to receive instructions from an input device; and
d. a processor configured to receive the instructions from the communication interface and to operate the cartridge controller for vaporization of the vaporization substance such that vapor is produced in accordance with the instructions,
wherein the apparatus is configured to remain disabled until an activation signal is received from the input device, wherein the activation signal is issued by a mobile application accessing an authentication mechanism of the input device once the authentication mechanism has determined at least in part from the cartridge information that the vapor is permitted to be produced in accordance with the instructions.

84. The apparatus of claim 83, wherein the cannabis-derived compound comprises THC or CBD.
